(19) United States
(12) Patent Application Publication
(10) Pub. No.: US 2005/0158262 A1
(43) Pub. Date: Jul. 21, 2005

(54) Cosmetic composition comprising a cationic agent, a polymer comprising a hetero atom and an oil, and cosmetic treatment process

(76) Inventor: Eric Parris, Clichy (FR)

Correspondence Address: Thomas L. Irving;FINNEGAN, HENDERSON , FARABOW,, GARRETT & DUNNER, L.L.P., 1300 I Street, N.W., Washington, DC, 20005-3315, US

(21) Appl. No.: 11/014,813

(22) Filed: Dec. 20, 2004

Related U.S. Application Data
(60) Provisional application No. 60/532,955, filed on Dec. 30, 2003.

(30) Foreign Application Priority Data
Dec. 19, 2003 (FR) 0351145

(51) Int. Cl. A61K 7/06

(52) U.S. Cl. 424/70.1

(57) ABSTRACT

The disclosure provides cosmetic compositions comprising, in a cosmetically acceptable medium, at least one cationic agent, at least one oil and at least one polymer comprising heteroatoms and fatty chains, and to cosmetic processes for treating keratin materials such as the hair. These compositions have improved and residual conditioning effects.

This application claims benefit of priority to U.S. Provisional Application No. 60/532,955, filed Dec. 30, 2003, which is hereby incorporated by reference.

The present disclosure provides cosmetic compositions, such as hair conditioners, comprising at least one cationic agent and at least one polymer comprising a heteroatom, combined with at least one oil, and to cosmetic processes for treating keratin materials such as the hair.

It is well known that hair that has been sensitized (i.e., damaged and/or embrittled) to varying degrees by atmospheric agents or by mechanical or chemical treatments, such as dyeing, bleaching and/or permanent-waving, is often difficult to disentangle, difficult to style, and lacks softness.

Improved hair conditioning is sought in the cosmetics field. As used herein, the term “conditioning” means the properties of easy disentangling, sheen, soft feel, and a smooth appearance and feel.

Cosmetic compositions containing cationic surfactants have been proposed for treating keratin materials such as the hair.

However, such compositions have drawbacks such as problems with rinseability, stability problems, difficulties in distributing them over the keratin materials, and insufficient cosmetic properties.

It has been recommended to use cationic polymers, cationic silicones, and cationic surfactants in compositions for washing and caring for keratin materials such as the hair, to facilitate the disentangling of the hair and to give it softness and suppleness. The use of cationic polymers and cations for this purpose has various drawbacks. On account of their high affinity for the hair, some of these polymers become deposited in substantial amount during repeated use, and lead to undesirable effects such as an unpleasant, laden feel, stiffening of the hair, and adhesion between the fibers that affects styling.

The current conditioning cosmetic compositions are not entirely satisfactory. Thus, there is a need for cosmetic compositions that have improved conditioning properties, including a smoother feel.

The use of a polymer with a terminal fatty chain and with a repeating unit containing at least one heteroatom is known in cosmetics, such as in the makeup field, as is described in French Patent Application No. 2,817,743. Cosmetic compositions whose fatty phase is gelled with such polymers have been described in French Patent Application No. 2,796,270, which describes solid lip compositions in the form of a stick.

However, these documents do not describe cosmetic compositions containing a cationic agent, much less, a cationic surfactant.

The Inventors have surprisingly discovered that the combination of at least one cationic agent, at least one polymer with a terminal or pendent fatty chain and with a repeating unit containing at least one heteroatom, and at least one oil, in a composition, for example, in non-detergent media with a low or zero concentration of washing surfactants, makes it possible to overcome these drawbacks.

Hair treated with these compositions is smooth, disentangles easily, is shiny, supple and individualized, and has a soft feel without a feeling of residues. The hair has a natural appearance and does not appear lank.

Without wishing to be bound by theory, it appears that the deposition of oil on the hair is significantly increased using these compositions, resulting in increased efficacy. This improvement is achieved, however, without giving the hair a greasy laden feel, which is usually the case when the amount of oil is increased.

Moreover, this conditioning effect may be resistant to rinsing.

Thus, according to the disclosure, novel cosmetic compositions, including non-washing compositions (i.e., leave-in compositions), are provided, comprising, in a cosmetically acceptable aqueous medium, at least one cationic agent, from 0.005% to 5% by weight of at least one oil, at least one polymer with a weight-average molecular mass of less than or equal to 1,000,000 comprising (a) a polymer skeleton with hydrocarbon-based repeating units comprising at least one heteroatom and (b) at least one pendent fatty chain and/or at least one terminal fatty chain, which are optionally functionalized, comprising from 6 to 120 carbon atoms and linked to these hydrocarbon-based units, and from 75% to 98% by weight of water relative to the total weight of the composition.

Cosmetic compositions are also provided which comprise, in a cosmetically acceptable aqueous medium, at least one cationic agent chosen from cationic surfactants, at least one oil and at least one polymer with a weight-average molecular mass of less than or equal to 1,000,000, comprising (a) a polymer skeleton with hydrocarbon-based repeating units comprising at least one heteroatom and (b) at least one pendent fatty chain and/or at least one terminal fatty chain, which are optionally functionalized, having from 6 to 120 carbon atoms and linked to these hydrocarbon-based units.

The disclosure also provides cosmetic processes for treating keratin materials, such as the hair, using the above-mentioned compositions.

The disclosure also provides methods of conditioning the hair using the compositions.

Other subjects, characteristics, aspects and advantages of the disclosure will emerge even more clearly on reading the description and the examples that follow.

The oil of the compositions may be prethickened with the above polymer, i.e., the oil and the polymer are optionally mixed together before being introduced into the composition.

The oil/polymer weight ratio may be greater than or equal to 50/50, for example, greater than or equal to 60/40, ranging from 60/40 to 99/1, for example, ranging from 80/20 to 99/1.

The oil, optionally prethickened, may be dispersed in the form of particles in the aqueous composition. The oil particles may have a number-average primary size ranging from 1 to 100 μm, for example from 5 to 30 μm and from 10 to 20 μm.

As used herein, the term “primary particle size” means the maximum dimension that it is possible to measure between two diametrically opposite points on an individual particle. The size may be determined, for example, by transmission electron microscopy, by measuring the specific surface area via the BET method, or alternatively by means of a laser granulometer.

The structuring polymer of the compositions is a solid that is undeformable at room temperature (25° C.) and atmospheric pressure (760 mmHg). It is insoluble in water and in the aqueous phase and it is capable of structuring the oil. The structuring polymer does not crystallize out of solution and the structuring of the liquid fatty phase is due to hydrogen bonding between two polymer molecules and/or between the molecules of the polymer and the molecules of the liquid fatty phase. The structuring polymer optionally contains no ionic groups.

U.S. Pat. No. 5,783,657 illustrates certain types of polymers that may be included in the compositions.

As used herein, the term “functionalized chains” means an alkyl chain comprising one or more functional or reactive groups chosen from amide, hydroxyl, ether, oxyalkylene, polyoxyalkylene, halogen (including fluoro and perfluoro groups), ester, siloxane, and polysiloxane groups. In addition, the hydrogen atoms of one or more fatty chains may be substituted at least partially with fluorine atoms.

The functionalized chains may be linked directly to the polymer skeleton or via an ester function or a perfluoro group.

As used herein, the term “polymer” means a compound comprising at least 2 repeating units, for example, at least 3 repeating units, which are identical.

As used herein, the term “hydrocarbon-based repeating units” means a unit comprising from 2 to 80 carbon atoms, such as from 2 to 60 carbon atoms, bearing hydrogen atoms and optionally oxygen atoms, which may be linear, branched or cyclic, and saturated or unsaturated. These repeating units each also comprise one or more heteroatoms that may be non-pendent and are in the polymer skeleton. The heteroatoms are chosen from nitrogen, sulfur, and phosphorus atoms and combinations thereof, optionally combined with one or more oxygen atoms. In some embodiments, the units comprise at least one nitrogen atom, for example, a non-pendent nitrogen atom. These units also may comprise a carbonyl group.

The units comprising a heteroatom are, for example, amide units forming a skeleton of the polyamide type, carbamate and/or urea units forming a polyurethane, polyurea, and/or polyurea-urethane skeleton, for example, amide units. The pendent chains are linked directly to at least one of the heteroatoms of the polymer skeleton. In one embodiment, the polymer comprises a polyamide skeleton and the end chains are linked to the polymer skeleton via a bonding group chosen from an ether, amine, urea, urethane, thioether, thioester, thiourea, thiourethane group, and a single bond.

The polymer may comprise silicone units or oxyalkylene units between the hydrocarbon-based units.

In addition, the polymers in the compositions may comprise a total number of fatty chains which represent from 40% to 98% of the total number of units comprising a heteroatom and of fatty chains, for example, from 50% to 95%. The nature and proportion of the units comprising a heteroatom depends on the nature of the liquid fatty phase and may be similar to the nature (polar or not) of the liquid fatty phase. Thus, the more the units containing a heteroatom are polar and in high proportion in the polymer, corresponding to the presence of several heteroatoms, the greater the affinity of the polymer for polar oils. Conversely, the more the units containing a heteroatom are non-polar, or even apolar, or the lower the proportion thereof, the greater the affinity of the polymer for apolar oils.

The polymer may be a polyamide with a weight-average molecular mass of less than 1,000,000, comprising (a) a polymer skeleton with amide repeating units and (b) optionally at least one pendent fatty chain and/or at least one terminal fatty chain, which is optionally functionalized, having from 6 to 120 carbon atoms and linked to the amide units.

The pendent fatty chains may be linked to at least one of the nitrogen atoms in the amide units of the polymer.

The fatty chains of this polyamide represent from 40% to 98% of the total number of units having a heteroatom and of fatty chains of the polymer, for example, from 50% to 95%.

The structuring polymer, e.g., the polyamide, in the compositions has a weight-average molecular mass of less than 1,000,000 and may be less than 500,000. The molecular mass may even be less than or equal to 100,000, for example, ranging from 1000 to 100,000; less than or equal to 50,000, for example, ranging from 1000 to 50,000, ranging from 1000 to 30,000, ranging from 2000 to 20,000, and ranging from 2000 to 10,000.

Structuring polymers which may be used in the compositions include polyamides optionally branched with pendent fatty chains and/or terminal fatty chains having from 6 to 120 carbon atoms, for example, from 12 to 120 carbon atoms and from 12 to 68 carbon atoms, the terminal fatty chains linked to the polyamide skeleton via bonding groups, for example, ester groups.

The polymers may be polymers resulting from a polycondensation between a dicarboxylic acid with at least 32 carbon atoms, for example, from 32 to 44 carbon atoms, and a diamine with at least 2 carbon atoms, for example, from 2 to 36 carbon atoms. Diacids include dimers of a fatty acid having at least 16 carbon atoms, for example, oleic acid, linoleic acid, and linolenic acid. Diamines include ethylenediamine, hexylenediamine or hexamethylenediamine. Polymers comprising one or 2 terminal carboxylic acid groups may be esterified with a monoalcohol with at least 4 carbon atoms, for example, from 10 to 36, 12 to 24, 16 to 24, such as 18 carbon atoms.

The polymers disclosed in U.S. Pat. No. 5,783,657 from Union Camp may be used. These polymers fall within formula (I) below: 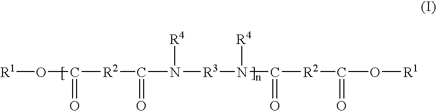
wherein:
n is a whole number of amide units such that the number of ester groups represents from 10% to 50% of the total number of ester and amide groups;
each R¹, which may be identical or different, is chosen from an alkyl or alkenyl group with at least 4 carbon atoms, for example from 4 to 24 carbon atoms;
each R², which may be identical or different, is a C₄ to C₄₂ hydrocarbon-based group, with the condition that 50% of the R² groups are a C₃₀ to C₄₂ hydrocarbon-based group;
each R³, which may be identical or different, is an organic group with at least 2 carbon atoms, hydrogen atoms and optionally one or more oxygen or nitrogen atoms; and
each R⁴, which may be identical or different, is chosen from a hydrogen atom, a C₁ to C₁₀ alkyl group, and a direct bond to R³ or to another R⁴, such that the nitrogen atom to which R³ and R⁴ are both attached forms part of a heterocyclic structure defined by R⁴—N—R³, with the condition that at least 50% of the groups R⁴ representing a hydrogen atom.

The ester groups of formula (I), which form part of the terminal and/or pendent fatty chains, may represent from 15% to 40% of the total number of ester and amide groups and better still from 20% to 35%. The variable n is an integer ranging from 1 to 10, such as from 1 to 5, for example, greater than 2. R¹ may be a C₁₂ to C₂₂ alkyl group, such as a C₁₆ to C₂₂ alkyl group. R² may be a C₁₀ to C₄₂ hydrocarbon-based group., for example, an alkylene group. In some embodiments, at least 50%, for example, at least 75% of the R² groups have from 30 to 42 carbon atoms. The other R² groups may be C₄ to C₁₉, for example, C₄ to C₁₂ or C₂ to C₁₂ hydrocarbon-based groups. R³ may be a C₂ to C₃₆ hydrocarbon-based group or a polyoxyalkylene group and R⁴ may be a hydrogen atom. In one embodiment, R³ is a C₂ to C₁₂ hydrocarbon-based group.

The hydrocarbon-based groups may be linear, cyclic, or branched, and saturated or unsaturated. Moreover, the alkyl and alkenyl groups may be linear or branched groups.

The structuring (or thickening) of the liquid fatty phase is obtained with the aid of one or more polymers of formula (I). In general, the polymers of formula (I) are in the form of mixtures of polymers, which may optionally further comprise a synthetic product corresponding to a compound of formula (I) in which n is 0, i.e., a diester.

Examples of structuring polymers which may be used in the compositions include the commercial products manufactured or sold by Arizona Chemical under the names Uniclear 80 and Uniclear 100. They are sold, respectively, in the form of an 80% (in terms of active material) gel in a mineral oil and a 100% (in terms of active material) gel. They have a softening point ranging from 88 to 94° C. These commercial products are a mixture of a copolymer of a C₃₆ diacid condensed with ethylenediamine, having an average molecular mass of about 6000. The terminal ester groups result from the esterification of the remaining terminal carboxylic acids with cetyl alcohol, stearyl alcohol, or mixtures thereof, also known as cetylstearyl alcohol.

Examples of structuring polymers which may be used in the compositions include polyamide resins resulting from the condensation of an aliphatic dicarboxylic acid and a diamine (including compounds having more than 2 carbonyl groups and 2 amine groups), the carbonyl and amine groups of adjacent individual units being condensed via an amide bond. These polyamide resins include those sold under the name Versamid® by the companies General Mills Inc. and Henkel Corp. (Versamid 930, 744, and 1655) and by the company Olin Mathieson Chemical Corp. under the brand name Onamid®, for example, Onamid S and C. In addition, Versamid® 930 or 744 may be used. These resins have a weight-average molecular mass ranging from 6000 to 9000. U.S. Pat. Nos. 3,645,705 and 3,148,125 disclose further information about these polymers.

The polyamides manufactured or sold by the company Arizona Chemical under the name Uni-Rez® (2658, 2931, 2970, 2621, 2613, 2624, 2665, 1554, 2623, and 2662) and the product sold under the reference Macromelt 6212 by the company Henkel may also be used. U.S. Pat. No. 5,500,209 discloses further information about these polymers.

It is also possible to use polyamide resins derived from plants, such as those disclosed in U.S. Pat. Nos. 5,783,657 and 5,998,570.

The structuring polymers in the compositions have a softening point of greater than 65° C., for example, greater than 70° C., and may be up to 190° C. They may have a softening point of less than 150° C., for example, ranging from 70 to 140° C., from 80 to 130° C., and even from 80 to 105° C. These polymers are generally non-waxy polymers. The low melting point of the structuring polymers makes them easier to use and limits the degradation of the liquid fatty phase, unlike polymers or compounds with higher softening points.

The polymers in the compositions may be polymers of formula (I). Because of the fatty chains, these polymers have good solubility in oils and thus result in compositions that are macroscopically homogeneous even with a high polymer content (e.g., at least 25%), unlike polymers not having a fatty chain.

Throughout the description, the softening point or melting point values may be determined by differential scanning calorimetry (DSC); the softening point (or melting point) corresponds to the melting peak when the temperature increase is 5 or 10° C./min.

The thickening of the fatty phase can be modified according to the nature of the polymers and their concentrations, and may be such that a viscosity ranging from 1000 to 250,000 cps, for example, from 10,000 to 50,000 cps at 25° C. is obtained, measured using a Rheomat 180 machine with a shear rate of 100 s⁻¹.

The polymers as defined above may be present in an amount from 0.005% to 20% by weight, for example, from 0.05% to 10% by weight, or even from 0.1% to 5% by weight, relative to the weight of the composition.

As used herein, the term “oil” means a liquid fatty substance that is insoluble in water at room temperature (25° C.) and atmospheric pressure (760 mmHg). The oily phase may comprise one or more mutually compatible oils.

As used herein, the term “water-insoluble” refers to a substance that has a solubility in pure water of less than 1% at 25° C. and at atmospheric pressure.

The oils used in the compositions have a dynamic viscosity at 25° C. of less than 1 Pa·s (1000 cps), for example, ranging from 10⁻³ to 0.1 Pa·s (1 to 100 cps). The dynamic viscosity is measured at 25° C. with a shear rate of 100 s⁻¹, for example, using the Rheomat RM 180 viscosimeter from Mettler.

The oils that may be used in the compositions are chosen from plant oils, mineral oils, synthetic oils, and fatty acid esters.

Examples of plant oils that may be used in the compositions include sweet almond oil, avocado oil, castor oil, olive oil, jojoba oil, sunflower oil, wheat germ oil, sesame seed oil, groundnut oil, grapeseed oil, soybean oil, rapeseed oil, safflower oil, coconut oil, maize oil, hazelnut oil, shea butter oil, palm oil, apricot kernel oil, and beauty-leaf oil.

Examples of mineral oils include paraffin oil and liquid petroleum jelly.

Examples of synthetic oils include polydecenes, squalane, poly(α-olefins), for example, isododecane, isohexadecane, transesterified plant oils, and fluoro oils.

Fatty acid esters may also be used, such as compounds of the formula RₐCOOR_{b} wherein Rₐ is a higher fatty acid residue having from 5 to 29 carbon atoms and R_{b} is a hydrocarbon-based chain having from 3 to 30 carbon atoms, such as purcellin oil (stearyl octanoate), isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate, and lactate.

The oils that may be used in the compositions include avocado oil, castor oil, olive oil, hydrogenated polydecene, isopropyl myristate, isononyl isononanoate, and liquid paraffin.

The oils as defined above may be present in an amount ranging from 0.005% to 30% by weight, for example, from 0.01% to 15% and from 0.01% to 5% by weight, relative to the weight of the composition.

In some embodiments, the oils may be present in an amount ranging from 0.01% to 3% by weight, for example, from 0.01% to 1% by weight, relative to the total weight of the composition.

The cationic agents may be chosen from cationic surfactants and cationic polymers and/or mixtures thereof, for example, cationic surfactants.

The cationic polymers that may be used in the compositions may be chosen from all those already known to improve the cosmetic properties of the hair, e.g., the polymers disclosed in EP Patent Application No. -337,354 and in French Patent Application Nos. 2,270,846; 2,383,660; 2,598,611; 2,470,596; and 2,519,863.

As used herein, the term “cationic polymer” means any polymer comprising cationic groups and/or groups that may be ionized into cationic groups.

Examples of cationic polymers that may be used in the compositions include polymers having units comprising primary, secondary, tertiary and/or quaternary amine groups that either may form part of the main polymer chain or may be borne by a side substituent directly attached thereto.

The cationic polymers may have a number-average molar mass ranging from 500 to 5×10⁶, for example, ranging from 10³ to 3×10⁶.

Cationic polymers that may be used in the compositions include polyamine, polyamino amide and polyquaternary ammonium polymers. These are known products.

Examples of polyamine, polyamido amide and polyquaternary ammonium polymers that may be used in the compositions include those disclosed in French Patent Nos. 2,505,348 and 2,542,997. These polymers include:
(1) homopolymers and copolymers derived from acrylic and methacrylic esters and amides and comprising at least one of the units of the following formulae: 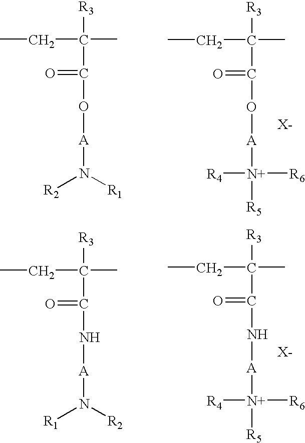
   wherein:
each R₃, which may be identical or different, is chosen from a hydrogen atom and a CH₃ radical;
each A, which may be identical or different, is chosen from a linear or branched alkyl group having from 1 to 6 carbon atoms, e.g., 2 or 3 carbon atoms, and a hydroxyalkyl group having from 1 to 4 carbon atoms;
each R₄, R₅, R₆, which may be identical or different, are chosen from an alkyl group having from 1 to 18 carbon atoms and a benzyl radical, e.g., an alkyl group having from 1 to 6 carbon atoms;
each R₁ and R₂, which may be identical or different, are chosen from hydrogen and an alkyl group having from 1 to 6 carbon atoms, e.g., methyl and ethyl; and
X is an anion derived from a mineral or organic acid, such as a methyl sulfate anion or a halide such as chloride or bromide.

The copolymers of family (1) above, can also comprise one or more units derived from comonomers which may be chosen from the family of acrylamides; methacrylamides; diacetone acrylamides; acrylamides; methacrylamides substituted on the nitrogen with lower alkyls(C₁-C₄); acrylic and methacrylic acids and esters thereof; vinyllactams such as vinylpyrrolidone and vinylcaprolactam; and vinyl esters.

The copolymers of family (1) above include:
copolymers of acrylamide and of dimethylaminoethyl methacrylate quaternized with dimethyl sulfate or with a dimethyl halide, such as the product sold under the name Hercofloc by Hercules;
copolymers of acrylamide and of methacryloyloxyethyltrimethylammonium chloride described, for example, in EP Patent Application 80,976 and sold under the name Bina Quat P 100 by Ciba Geigy;
copolymers of acrylamide and of methacryloyloxyethyltrimethylammonium methosulfate sold under the name Reten by Hercules;
quaternized or nonquaternized vinylpyrrolidone/dialkylaminoalkyl acrylate or methacrylate copolymers, such as the products sold under the name Gafquat by the company ISP, for example, Gafquat 734 and Gafquat 755, and Copolymers 845, 958 and 937. These polymers are disclosed in French Patent Nos. 2,077,143 and 2,393,573;
dimethylaminoethyl methacrylate/vinylcaprolactam/vinylpyrrolidone terpolymers, such as the product sold under the name Gaffix® VC 713 by ISP;
vinylpyrrolidone/methacrylamidopropyldimethylamine copolymers sold under the name Styleze CC 10 by ISP; and
quaternized vinylpyrrolidone/dimethylaminopropylmethacrylamide copolymers, such as the product sold under the name Gafquat HS 100 by the company ISP.

Examples of polymers that may be used in the compositions also include (2) cationic polysaccharides, such as cationic celluloses and cationic galactomannan gums. Cationic polysaccharides include cellulose ether derivatives comprising quaternary ammonium groups, cationic cellulose copolymers or cellulose derivatives grafted with a water-soluble quaternary ammonium monomer, and cationic galactomannan gums.

The cellulose ether derivatives comprising quaternary ammonium groups, are disclosed in French Patent No. 1,492,597, for example, the polymers sold under the names JR (e.g., JR 400, JR 125, and JR 30M) and LR (e.g., LR 400 and LR 30M) by the company Amerchol. These polymers are defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are disclosed in U.S. Pat. No. 4,131,576, for example hydroxyalkylcelluloses such as hydroxymethyl-, hydroxyethyl- and hydroxypropylcelluloses grafted with a methacryloylethyltrimethylammonium, methacrylamidopropyltrimethylammonium or dimethyldiallylammonium salt.

Such copolymers include the products sold under the names “Celquat L 200” and “Celquat H 100” by the company National Starch.

The cationic galactomannan gums are disclosed in U.S. Pat. Nos. 3,589,578 and 4,031,307, such as guar gums having trialkylammonium cationic groups. For example, guar gums modified with a salt, e.g., chloride of 2,3-epoxypropyltrimethylammonium may be used.

Such products include those sold under the names Jaguar C13 S, Jaguar C 15, Jaguar C 17, and Jaguar C 162 by the company Rhodia.

Examples of polymers that may be used in the compositions also include (3) polymers comprising piperazinyl units and divalent alkylene or hydroxyalkylene radicals having straight or branched chains, optionally interrupted by oxygen, sulfur, or nitrogen atoms or by aromatic or heterocyclic rings, as well as the oxidation and/or quaternization products of these polymers. Such polymers are described, for example, in French Patent Nos. 2,162,025 and 2,280,361.

Further examples of polymers that may be used in the compositions include (4) water-soluble polyamino amides prepared by the polycondensation of an acidic compound with a polyamine. These polyamino amides may be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide, or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide. The polyamino amides may be alkylated or, if they comprise one or more tertiary amine functions, may be quaternized. Such polymers are described, for example, in French Patent Nos. 2,252,840 and 2,368,508.

Polymers that may be used in the compositions also include (5) polyaminoamide derivatives resulting from the condensation of polyalkylene polyamines with polycarboxylic acids followed by alkylation with difunctional agents. Examples include adipic acid/dialkylaminohydroxyalkyldialkylenetriamine polymers in which the alkyl radical has from 1 to 4 carbon atoms, for example, methyl, ethyl, and propyl. Such polymers are disclosed in French Patent No. 1,583,363.

These derivatives include the adipic acid/dimethylamino-hydroxypropyl/diethylenetriamine polymers sold under the name Cartaretine F, F4, and F8 by the company Sandoz.

In addition, polymers that may be used in the compositions also include (6) polymers obtained by reaction of a polyalkylene polyamine having two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid may range from 0.8:1 to1.4:1; the polyamino amide resulting therefrom may be reacted with epichlorohydrin in a molar ratio of epichlorohydrin relative to the secondary amine group of the polyamino amide may range from 0.5:1 to 1.8:1. Such polymers are disclosed in U.S. Pat. Nos. 3,227,615 and 2,961,347. Examples of these polymers include Hercosett 57 by the company Hercules Inc. and PD 170 and Delsette 101 by the company Hercules (adipic acid/epoxypropyl/diethylenetriamine copolymer).

Polymers that may be used in the compositions also include (7) cyclopolymers of alkyldiallylamine or of dialkyldiallylammonium, such as the homopolymers or copolymers having, as main constituent of the chain, units corresponding to formulae (II) and (III): 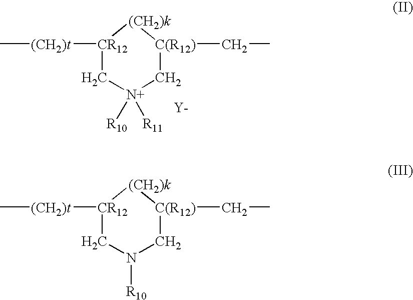
wherein k and t are equal to 0 or 1 and the sum of (k+t) is equal to 1;
each R₁₂ is chosen from a hydrogen atom and a methyl;
each R₁₀ and R₁₁, which may be identical or different, is chosen from an alkyl group having from 1 to 6 carbon atoms, a hydroxyalkyl group in which the alkyl group may have 1 to 5 carbon atoms, and a lower C₁-C₄ amidoalkyl group, or R₁₀ and R₁₁ can denote, together with the nitrogen atom to which they are attached, heterocyclic groups such as piperidyl or morpholinyl; and
Y⁻ is an anion such as bromide, chloride, acetate, borate, citrate, tartrate, bisulfate, bisulfite, sulfate, or phosphate.

These polymers are disclosed in French Patent No. 2,080,759 and in its Certificate of Addition 2,190,406. For example, each R₁₀ and R₁₁, which may be identical or different, may be an alkyl group having from 1 to 4 carbon atoms.

These polymers include the dimethyldiallylammonium chloride homopolymer sold under the name Merquat 100 by the company Nalco (and its homologues of low weight-average molar mass) and copolymers of diallyidimethylammonium chloride and of acrylamide, sold under the name Merquat 550.

Polymers that may be used in the compositions also include (8) quaternary diammonium polymers comprising repeating units corresponding to formula (IV): 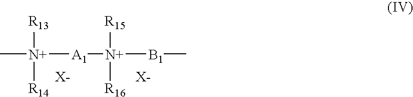
wherein:
each R₁₃, R₁₄, R₁₅ and R₁₆, which may be identical or different, are chosen from aliphatic, alicyclic, and arylaliphatic radicals having from 1 to 20 carbon atoms and lower hydroxyalkylaliphatic radicals;
each R₁₃, R₁₄, R₁₅ and R₁₆, which may be identical or different, together or separately, constitute, with the nitrogen atoms to which they are attached, heterocycles optionally having a second heteroatom other than nitrogen; or each R₁₃, R₁₄, R₁₅ and R₁₆, which may be identical or different, are chosen from a linear or branched C₁—C₆ alkyl radical substituted with a nitrile, ester, acyl or amide group or a group —CO—O—R₁₇-D or —CO—NH—R₁₇-D wherein R₁₇ is an alkylene group and D is a quaternary ammonium group;
each A₁ and B₁ are chosen from polymethylene groups having from 2 to 20 carbon atoms, which may be linear or branched, saturated or unsaturated, and which may comprise, linked to or intercalated in the main chain, one or more aromatic rings or one or more oxygen atoms; sulfur atoms; and sulfoxide, sulfone, disulfide, amino, alkylamino, hydroxyl, quaternary ammonium, ureido, amide or ester groups, and
X⁻ denotes an anion derived from an inorganic or organic acid.

Optionally, A₁, R₁₃ and R₁₅ can form, together with the two nitrogen atoms to which they are attached, a piperazine ring; in addition, if A₁ denotes a linear or branched, saturated or unsaturated alkylene or hydroxyalkylene radical, B₁ may also denote a group (CH₂)ₙ—CO-D-OC—(CH₂)ₙ—; wherein D is chosen from:
(a) a glycol residue of formula: —O-Z-O—, wherein Z is chosen from a linear or branched hydrocarbon-based radical or a group corresponding to one of the following formulae:
   —(CH₂—CH₂—O)ₓ—CH₂—CH₂
   —[CH₂—CH(CH₃)—O]_{y}—CH₂—CH(CH₃)—
   wherein x and y denote an integer ranging from 1 to 4, representing a defined and unique degree of polymerization or any number ranging from 1 to 4 representing an average degree of polymerization;
(b) a bis-secondary diamine residue such as a piperazine derivative;
(c) a bis-primary diamine residue of formula: —NH—Y—NH—, wherein Y is chosen from a linear or branched hydrocarbon-based radical, and the divalent radical
   —CH₂—CH₂—S—S—CH₂—CH₂—;
(d) a ureylene group of formula: —NH—CO—NH—; and
each n, which may be identical or different, is an integer ranging from 2 to 20, for example, 2 to 6, and
X⁻ may be an anion such as chloride or bromide.

These polymers generally have a number-average molar mass ranging from 1000 to 100,000.

Polymers of this type are described in French Patent Nos. 2,320,330; 2,270,846; 2,316,27; 2,336,434; and 2,413,907; and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

For example, polymers comprising repeating units corresponding to formula (V): 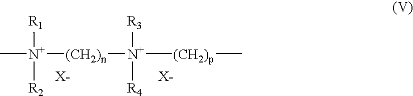
may be used wherein
each R₁, R₂, R₃ and R₄, which may be identical or different, is chosen from an alkyl or hydroxyalkyl radical having from 1 to 4 carbon atoms,
n and p are each integers ranging from 2 to 20, and
X⁻ is an anion derived from an inorganic or organic acid.

Hexadimethrine chloride (according to the INCI (CTFA) nomenclature) is an example of a compound of formula (V) wherein R₁, R₂, R₃ and R₄ each represent a methyl radical, n is 3, p is 6, and X is Cl.

Polymers that may be used in the compositions also include
(9) polyquaternary ammonium polymers comprising units of formula (VI): 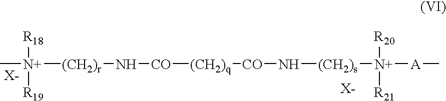
   wherein each R₁₈, R₁₉, R₂₀ and R₂₁, which may be identical or different, is chosen from a hydrogen atom and a methyl, ethyl, propyl, β-hydroxyethyl, β-hydroxypropyl and —CH₂CH₂(OCH₂CH₂)ₚOH radical,
p is equal to 0 or to an integer ranging from 1 to 6, with the proviso that R₁₈, R₁₉, R₂₀ and R₂₁ are not all hydrogen atoms,
each r and s, which may be identical or different, are integers ranging from 1 to 6,
q is equal to 0 or is an integer ranging from 1 to 34,
X⁻ is an anion such as a halide, and
A is chosen from a dihalide radical and —CH₂—CH₂—O—CH₂—CH₂—.

Such compounds are disclosed in EP Patent Application No. 122,324. These compounds include the products Mirapol® A 15, Mirapol® AD1, Mirapole AZ1, and Mirapol® 175 sold by the company Miranol.

Polymers that may be used in the compositions also include (10) quaternary polymers of vinylpyrrolidone and of vinylimidazole, for example the products sold under the names Luviquat® FC 905, FC 550, and FC 370 by the company BASF.

Polymers that may be used in the compositions also include (11) polyamines, for example Polyquart® H sold by Cognis, referenced under the name Polyethylene Glycol (15) Tallow Polyamine in the CTFA dictionary.

Polymers that may be used in the compositions also include (12) crosslinked or noncrosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound having olefinic unsaturation, such as methylenebisacrylamide. A crosslinked acrylamide/methacryloyloxyethyltrimethylammonium chloride copolymer (20/80 by weight) in the form of a dispersion having 50% by weight of the copolymer in mineral oil may be used. This dispersion is sold under the name Salcare® SC 92 by the company Ciba. A crosslinked methacryloyloxyethyltrimethylammonium chloride homopolymer comprising about 50% by weight of the homopolymer in mineral oil or in a liquid ester can also be used. These dispersions are sold under the names Salcare® SC 95 and Salcare® SC 96 by the company Ciba.

Other cationic polymers that can be used in compositions include cationic proteins, cationic protein hydrolysates, polyalkyleneimines, for example, polyethyleneimines, polymers comprising vinylpyridine or vinylpyridinium units, condensates of polyamines and of epichlorohydrin, quaternary polyureylenes, and chitin derivatives.

The cationic polymers mentioned above include quaternary cellulose ether derivatives such as the products sold under the name JR 400 by the company Amerchol, cationic cyclopolymers, for example, the dimethyldiallylammonium chloride homopolymers and copolymers sold under the names Merquat 100, Merquat 550, and Merquat S by the company Nalco, quaternary polymers of vinylpyrrolidone and of vinylimidazole, optionally crosslinked homopolymers or copolymers of methacryloyloxy(C₁-C₄)alkyltri(C₁-C₄)alkylammonium salts, and mixtures thereof.

The cationic polymers are generally present in concentrations ranging from 0.001% to 20%, such as from 0.5% to 10% and from 0.1% to 5%, by weight relative to the total weight of the composition.

The compositions may comprise one or more known cationic surfactants, such as optionally polyoxyalkylenated primary, secondary, and tertiary fatty amine salts, quaternary ammonium salts, and mixtures thereof.

Examples of quaternary ammonium salts include:
those of general formula (V) below: 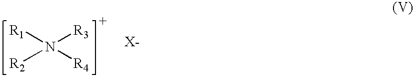
   wherein R₁, R₂, R₃, and R₄, which may be identical or different, are chosen from linear and branched aliphatic radicals having from 1 to 30 carbon atoms, aromatic radicals such as aryl and alkylaryl. The aliphatic radicals may comprise heteroatoms such as oxygen, nitrogen, and sulfur as well as halogens. The aliphatic radicals are chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate, and hydroxyalkyl radicals, having from about 1 to 30 carbon atoms; X⁻ is an anion chosen from halides, phosphates, acetates, lactates, (C₂-C₆) alkyl sulfates, and alkyl- or alkylaryl-sulfonates;
quaternary ammonium salts of imidazoline, for example, those of formula (VI) below: 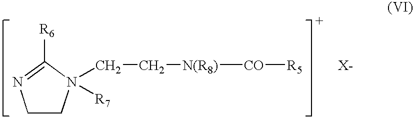
   wherein R₅ is chosen from an alkenyl or alkyl radical having from 8 to 30 carbon atoms, for example, fatty acid derivatives of tallow or of coconut; R₆ is chosen from a hydrogen atom, a C₁-C₄ alkyl radical, and an alkenyl or alkyl radical having from 8 to 30 carbon atoms; R₇ is chosen from a C₁-C₄ alkyl radical; R₈ is chosen from a hydrogen atom and a C₁-C₄ alkyl radical; and X is an anion chosen from halides, phosphates, acetates, lactates, alkyl sulfates, alkyl sulfonates, and alkylaryl sulfonates. For example, R₅ and R₆ may be a mixture of alkenyl or alkyl radicals having from 12 to 21 carbon atoms, such as fatty acid derivatives of tallow, R₇ may be methyl, and R₈ may be hydrogen. Such a product is, for example, Quaternium-27 (CTFA 1997) or Quaternium-83 (CTFA 1997), which are sold under the names Rewoquat® W75, W90, W75PG and W75HPG by the company Witco;
diquaternary ammonium salts of formula (VII): 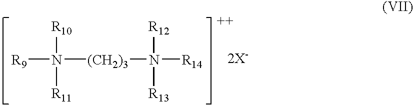
   wherein R₉ is an aliphatic radical having from about 16 to 30 carbon atoms; each R₁₀, R₁₁, R₁₂, R₁₃ and R₁₄, which may be identical or different, are chosen from hydrogen and an alkyl radical having from 1 to 4 carbon atoms; and each X⁻ is an anion chosen from the group of halides, acetates, phosphates, nitrates, ethyl sulfates and methyl sulfates. Such diquaternary ammonium salts include propanetallowdiammonium dichloride;
quaternary ammonium salts comprising at least one ester function, such as those of formula (VIII) below: 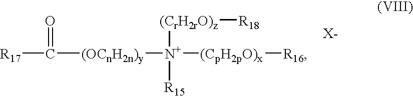
   wherein R₁₅ is chosen from C₁-C₆ alkyl radicals and C₁-C₆ hydroxyalkyl or dihydroxyalkyl radicals;
R₁₆ is chosen from:
   a radical 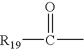
   linear or branched, saturated or unsaturated C₁-C₂₂ hydrocarbon-based radicals R₂₀, and
   a hydrogen atom;
R₁₈ is chosen from:
   a radical 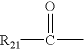
   linear or branched, saturated or unsaturated C₁-C₆ hydrocarbon-based radicals R₂₂, and
   a hydrogen atom;
each R₁₇, R₁₉ and R₂₁, which may be identical or different, are chosen from linear or branched, saturated or unsaturated, C₇-C₂₁ hydrocarbon-based radicals;
r, n and p, which may be identical or different, are integers ranging from 2 to 6;
y is an integer ranging from 1 to 10;
x and z, which may be identical or different, are integers ranging from 0 to 10;
X⁻ is a simple or complex organic or inorganic anion;
with the proviso that when the sum of (x+y+z) is from 1 to 15 and x is 0, then R₁₆ denotes R₂₀ and that when z is 0, then R₁₈ denotes R₂₂.

The alkyl radicals R₁₅ may be linear or branched, for example, linear.

R₁₅ may be chosen from a methyl, ethyl, hydroxyethyl, and dihydroxypropyl radical, for example, a methyl or ethyl radical.

In some embodiments, the sum of (x+y+z) may range from 1 to 10.

In some embodiments, when R₁₆ is a hydrocarbon-based radical R₂₀, it may be long and have from 12 to 22 carbon atoms, or be short and have from 1 to 3 carbon atoms.

In some embodiments, when R₁₈ is a hydrocarbon-based radical R₂₂, it may have 1 to 3 carbon atoms.

In some embodiments, R₁₇, R₁₉ and R₂₁, which may be identical or different, are chosen from linear or branched, saturated or unsaturated C₁₁-C₂₁ hydrocarbon-based radicals, for example, from linear and branched, saturated and unsaturated C₁₁-C₂₁ alkyl and alkenyl radicals.

In some embodiments, x and z, which may be identical or different, are 0 or 1.

In some embodiments, y is equal to 1.

In some embodiments, r, n, and p, which may be identical or different, are equal to 2 or 3, for example, equal to 2.

The anion X may be a halide such as chloride, bromide or iodide or a C₁-C₄ alkyl sulfate, e.g., methyl sulfate. Methanesulfonate, phosphate, nitrate, tosylate, an anion derived from an organic acid, such as acetate or lactate, or any other anion that is compatible with the ammonium having an ester function may also be used.

In some embodiments, the anion X⁻ is chloride or methyl sulfate.

Ammonium salts of formula (IV) may be used wherein:
R₁₅ is a methyl or ethyl radical;
x and y are equal to 1;
z is equal to 0 or 1;
r, n and p are equal to 2;
R₁₆ is chosen from:
a radical 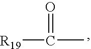
methyl, ethyl or C₁₄-C₂₂ hydrocarbon-based radicals, and
a hydrogen atom;
R₁₈ is chosen from:
a radical 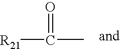
a hydrogen atom; and
R₁₇, R₁₉ and R₂₁, which may be identical or different, are chosen from linear and branched, saturated and unsaturated, C₁₃-C₁₇ hydrocarbon-based radicals, such as linear or branched, saturated or unsaturated C₁₃-C₁₇ alkyl and alkenyl radicals.

The hydrocarbon-based radicals are optionally linear.

Examples of compounds of formula (VIII) include the salts (e.g., chloride and methyl sulfate salts) of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethylammonium, and mixtures thereof. The acyl radicals optionally have from 14 to 18 carbon atoms and may be derived from a plant oil, for example palm oil or sunflower oil. When the compound has several acyl radicals, these radicals may be identical or different.

These products may be obtained, for example, by direct esterification of optionally oxyalkylenated triethanolamine, triisopropanolamine, alkyldiethanolamine or alkyldiisopropanolamine onto fatty acids and onto mixtures of fatty acids of plant or animal origin, or by transesterification of methyl esters thereof. This esterification is followed by a quaternization using an alkylating agent such as an alkyl halide (e.g., a methyl or ethyl halide), a dialkyl sulfate (e.g., dimethyl or diethyl sulfate), methyl methanesulfonate, methyl para-toluenesulfonate, glycol chlorohydrin, or glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

The compositions optionally comprise a mixture of quaternary ammonium mono-, di- and triester salts with a weight majority of diester salts.

Examples of mixtures of ammonium salts that may be used include the mixture having from 15% to 30% by weight of acyloxyethyl-dihydroxyethyl-methyl-ammonium methyl sulfate, from 45% to 60% of diacyloxyethyl-hydroxyethyl-methylammonium methyl sulfate, and from 15% to 30% of triacyloxyethyl-methylammonium methyl sulfate, the acyl radicals having from 14 to 18 carbon atoms and being derived from optionally partially hydrogenated palm oil.

It is also possible to use the ammonium salts having at least one ester function described in U.S. Pat. Nos. 4,874,554 and 4,137,180.

The quaternary ammonium salts that may be used include those corresponding to formula (V), such as (a) tetraalkylammonium chlorides, for example dialkyldimethylammonium and alkyltrimethylammonium chlorides in which the alkyl radical has from about 12 to 22 carbon atoms, e.g., behenyltrimethylammonium, distearyidimethyl-ammonium, cetyltrimethylammonium, and benzyldimethylstearylammonium chloride, and (b) palmitylamidopropyltrimethylammonium chloride and stearamidopropyldimethyl(myristyl acetate)ammonium chloride sold under the name Ceraphyl® 70 by the company Van Dyk.

The cationic surfactants may be used in the compositions may be chosen from quaternary ammonium salts, for example, behenyltrimethylammonium chloride, cetyltrimethylammoniumchloride, quaternium-83, behenylamidopropyl-2,3-dihydroxypropyldimethylammonium chloride, and palmitylamidopropyltrimethylammonium chloride.

The compositions may comprise the cationic surfactants in an amount ranging from 0.05% to 10% by weight, for example, from 0.1% to 8%, from 0.2% to 6%, and from 0.3% to 3% by weight, relative to the total weight of the composition.

The disclosed compositions may optionally comprise surfactants other than cationic surfactants, such as nonionic or amphoteric surfactants.

The additional surfactants are generally present in an amount ranging from 0.1% to 10% by weight, for example, from 0.5% to 8% and from 1% to 5% by weight, relative to the total weight of the composition.

The compositions optionally comprise at least one surfactant chosen from nonionic surfactants.

Nonionic surfactants are known compounds, for example, in “Handbook of Surfactants” by M. R. Porter, published by Blackie & Son (Glasgow and London), 1991, pp. 116-178. Nonionic surfactants include, but are not limited to, polyethoxylated, polypropoxylated, and polyglycerolated fatty alcohols; polyethoxylated, polypropoxylated, and polyglycerolated fatty α-diols; polyethoxylated, polypropoxylated, and polyglycerolated fatty alkylphenols; and polyethoxylated, polypropoxylated, and polyglycerolated fatty acids. These compounds comprise fatty chains having, for example, 8 to 18 carbon atoms and the number of ethylene oxide or propylene oxide groups may range from 2 to 50 and the number of glycerol groups may range from 2 to 30. Examples also include copolymers of ethylene oxide and of propylene oxide; condensates of ethylene oxide and of propylene oxide with fatty alcohols; polyethoxylated fatty amides having from 2 to 30 mol of ethylene oxide; polyglycerolated fatty amides having on average 1 to 5, e.g., 1.5 to 4, glycerol groups; oxyethylenated fatty acid esters of sorbitan having from 2 to 30 mol of ethylene oxide; fatty acid esters of sucrose; fatty acid esters of polyethylene glycol; alkylpolyglycosides; N-alkylglucamine derivatives; amine oxides such as (C₁₀-C14)-alkylamine oxides; and N-acylaminopropylmorpholine oxides. For example, alkylpolyglycosides may be used as nonionic surfactants.

The non-washing (non-detergent) compositions optionally comprise less than 4% by weight, e.g., less than 1% by weight, of anionic detergent surfactants relative to the total weight of the composition.

The compositions also optionally comprise at least one conditioning agent.

As used herein, the term “conditioner” means any agent whose function is to improve the cosmetic properties of keratin materials such as the hair, including the softness, smoothness, disentangling, feel, and static electricity.

The conditioners may be soluble or insoluble in water.

The conditioners include natural and synthetic waxes, ceramide compounds, carboxylic acid esters, silicones, anionic polymers, nonionic polymers, cationic polymers, amphoteric polymers, cationic proteins, cationic protein hydrolysates usually used in cosmetic or dermatological compositions, and mixtures thereof.

The insoluble conditioning agents may be solid, liquid, or pasty at room temperature (25° C.) and at atmospheric pressure, and may be in the form of oils, waxes, resins or gums.

The insoluble conditioning agents are optionally dispersed in the compositions in the form of particles generally having a number-average size ranging from 2 nanometers and 100 microns, for example, from 30 nanometers to 20 microns, measured with a granulometer.

The conditioning agents may be present in the compositions in an amount ranging from 0.01% to 20% by weight, e.g., from 0.05% to 10% or from 0.1% to 5% by weight, relative to the total weight of the composition.

The cosmetically acceptable medium may be aqueous and may comprise water or a mixture of water and a cosmetically acceptable solvent such as a C₁-C₄ lower alcohol, for example, ethanol, isopropanol, tert-butanol, and n-butanol; polyols, for example, propylene glycol and glycerol; polyol ethers; C₅-C₁₀ alkanes; and mixtures thereof. The solvents may be chosen from glycerol and propylene glycol.

The cosmetically acceptable medium, which may be aqueous, represents from 30% to 98% by weight, for example, from 80% to 98% by weight, relative to the total weight of the composition.

The compositions may comprise from 75% to 99% by weight, for example, from 80% to 98% by weight, of water relative to the total weight of the composition.

The solvents may be present in concentrations ranging from 0.5% to 30% by weight relative to the total weight of the composition.

The pH of the compositions ranges from 2 to 8, for example, from 3 to 7.5.

The compositions may also comprise standard additives that are well known in the art, such as anionic, nonionic, and amphoteric polymers; nonpolymeric thickeners, for example, acids and electrolytes; opacifiers; nacreous agents; vitamins; provitamins such as panthenol; waxes such as plant waxes; natural and synthetic ceramides; fragrances; colorants; organic and mineral particles; preserving agents; and pH stabilizers.

A person skilled in the art will take care to select the optional additives and the amount thereof such that they do not harm the properties of the compositions.

The additives may be present in the compositions in an amount ranging from 0% to 20% by weight relative to the total weight of the composition.

The compositions may be in the form of a rinse-out or leave-in conditioner; permanent waving, relaxing, dyeing or bleaching compositions; or alternatively in the form of rinse-out compositions to be applied before a dyeing, bleaching, permanent-waving or relaxing operation or alternatively between the two steps of a permanent-waving or relaxing operation.

The compositions may be used, for example, as conditioners, care products, deep-down care masks, and scalp treatment lotions or creams. These compositions may be rinse-out or leave-in compositions.

According to one embodiment, the composition may be used as a conditioner, for example, on fine hair. This conditioner may be a rinse-out or leave-in conditioner, for example, a rinse-out conditioner.

The cosmetic compositions may be in the form of a gel, a milk, a cream, an emulsion, fluid or thickened lotions, or a foam, and may be used for the skin, the nails, the eyelashes, the lips and, for example, the hair.

The compositions may be packaged in various forms, for example in vaporizers, pump-dispenser bottles or in aerosol containers in order to dispense the composition in vaporized form or in the form of a mousse. Such packaging forms are used, for example, when it is desired to obtain a spray, a lacquer or a mousse for treating the hair.

The present disclosure also provides cosmetic processes for treating keratin materials such as, for example, the skin and the hair, which comprises applying an effective amount of a cosmetic composition as described above to the keratin materials, and optionally rinsing it off after optionally leaving it to act for a period of time.

The rinsing is performed, for example, with water.

Thus, this process allows holding of the hairstyle and treatment, conditioning and care of the hair or any other keratin material.

The invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLES 1 AND 2

The following rinse-out conditioning compositions were prepared:

| Composition | 1 | 2 |
|---|---|---|
| Inulin (Raftiline HP - Orafti) | 15 g | — |
| Polyquaternium-37 (Salcare SC 95 - Ciba) | 0.5 g | — |
| Cetylstearyl alcohol | — | 2 g |
| Cetyl esters (Spermwax vegetal - Robeco) | — | 0.25 g |
| Behentrimonium chloride (Genamin KDMP - | — | 1.4 g |
| Clariant) | | |
| Mixture (95/5 by weight) of copolymer of a C36 | — | 1 g |
| diacid condensed with ethylenediamine (Uniclear | | |
| 100 VG from Arizona Chemical) and of | | |
| hydrogenated polydecene (Ceraflow E from | | |
| Shamrock) | | |
| Preserving agent | qs | qs |
| pH agent qs | pH = 7 | pH = 3.5 |
| Water | qs 100 g | qs 100 g |

The compositions were applied to sensitized hair. After a leave-in time of 3 minutes, the hair was rinsed. The hair disentangled easily and was very smooth.

1. A cosmetic composition comprising, in a cosmetically acceptable aqueous medium:
at least one cationic agent;
from 0.005% to 5% by weight of at least one oil;
at least one polymer with a weight-average molecular mass of less than or equal to 1,000,000, comprising (a) a polymer skeleton comprising hydrocarbon-based repeating units comprising at least one heteroatom, and (b) at least one chain chosen from a pendant and a terminal fatty chain which is optionally functionalized, having from 6 to 120 carbon atoms and linked to the hydrocarbon-based units; and
from 75% to 98% by weight of water relative to the total weight of the composition.

2. The composition according to claim 1, wherein the weight-average molecular mass of the at least one polymer is less than or equal to 500,000.

3. The composition according to claim 2, wherein the weight-average molecular mass of the at least one polymer is less than or equal to 100,000.

4. The composition according to claim 3, wherein the weight-average molecular mass of the at least one polymer ranges from 1000 to 30,000.

5. The composition according to claim 4, wherein the weight-average molecular mass of the at least one polymer ranges from 2000 to 10,000.

6. The composition according to claim 1, wherein the hydrocarbon-based repeating units comprise at least one nitrogen atom.

7. The composition according to claim 6, wherein the hydrocarbon-based repeating units are amide groups.

8. The composition according to claim 7, wherein the at least one fatty chain is pendant and linked directly to the nitrogen atom of the amide groups.

9. The composition according to claim 1, wherein the at least one fatty chain represents from 40% to 98% of the total number of hydrocarbon-based repeating units and of fatty chains.

10. The composition according to claim 1, wherein the fatty chains represent from 50% to 95% of the total number of heteroatom-containing units and fatty chains.

11. The composition according to claim 1, wherein the at least one fatty chain is pendent and linked directly to at least one heteroatom.

12. The composition according to claim 1, wherein the at least one fatty chain is terminal and linked to the polymer skeleton via a bonding group.

13. The composition according to claim 12, wherein the bonding group is an ester group.

14. The composition according to claim 1, wherein the at least one fatty chain has from 12 to 68 carbon atoms.

15. The composition according to claim 1, wherein the at least one polymer is chosen from the polymers of formula (I): 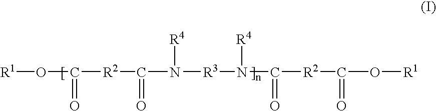
and mixtures thereof wherein:
n is a number of amide units such that the number of ester groups represents from 10% to 50% of the total number of ester and amide groups;
each R¹ is chosen from an alkyl and alkenyl group having at least 4 carbon atoms;
each R² is chosen from a C₄ to C₄₂ hydrocarbon-based group, with the condition that at least 50% of the R² groups are a C₃₀ to C₄₂ hydrocarbon-based group;
each R³ is chosen from an organic group comprising at least 2 carbon atoms, hydrogen atoms and optionally one or more oxygen or nitrogen atoms; and
each R⁴ is chosen from a hydrogen atom, a C₁ to C₁₀ alkyl group, and a direct bond to R³ or to another R⁴, such that the nitrogen atom to which R³ and R⁴ are both attached forms part of a heterocyclic structure defined by R⁴—N—R³, with at least 50% of the groups R⁴ representing a hydrogen atom.

16. The composition according to claim 15, wherein each R¹ is a C₁₂ to C₂₂ alkyl group.

17. The composition according to claim 15, wherein each R² is a C₃₀ to C₄₂ hydrocarbon-based group.

18. The composition according to claim 1, wherein the polymer is present in an amount ranging from 0.005% to 20% by weight relative to the total weight of the composition.

19. The composition according to claim 18, wherein the polymer is present in an amount ranging from 0.05% to 10% by weight relative to the total weight of the composition.

20. The composition according to claim 1, wherein the at least one oil is chosen from plant oils, mineral oils, synthetic oils, and fatty acid esters.

21. The composition according to claim 20, wherein the at least one oil is chosen from sweet almond oil, avocado oil, castor oil, olive oil, jojoba oil, sunflower oil, wheat germ oil, sesame seed oil, groundnut oil, grapeseed oil, soybean oil, rapeseed oil, safflower oil, coconut oil, maize oil, hazelnut oil, shea butter oil, palm oil, apricot kernel oil and beauty-leaf oil, paraffin oil, liquid petroleum jelly, polydecenes, squalane, poly(α-olefins), compounds of formula RₐCOOR_{b} wherein Rₐ is a higher fatty acid residue having from 5 to 29 carbon atoms and R_{b} is a hydrocarbon-based chain having from 3 to 30 carbon atoms.

22. The composition according to claim 21, wherein the at least one oil is a poly(α-olefin) chosen from isododecane, isohexadecane, transesterified plant oils, and fluoro oils.

23. The composition according to claim 21, wherein the at least one oil is chosen from avocado oil, castor oil, olive oil, isohexadecane, polydecene, isopropyl myristate, isononyl isononanoate, and liquid paraffin.

24. The composition according to claim 1, wherein the at least one oil is present in an amount ranging from 0.01% to 3% by weight relative to the total weight of the composition.

25. The composition according to claim 24, wherein the at least one oil is present in an amount ranging from 0.01% to 1% by weight relative to the total weight of the composition.

26. The composition according to claim 1, wherein the at least one oil is prethickened with the polymer.

27. The composition according to claim 1, wherein the weight ratio of the at least one oil to polymer is greater than or equal to 50/50.

28. The composition according to claim 27, wherein the weight ratio of the at least one oil to polymer is greater than or equal to 60/40.

29. The composition according to claim 28, wherein the weight ratio of the at least one oil to polymer ranges from 60/40 to 99/1.

30. The composition according to claim 1, wherein the at least one oil has a number-average primary size ranging from 1 to 100 μm.

31. The composition according to claim 30, wherein the at least one oil has a number-average primary size ranging from 5 to 30 μm.

32. The composition according to claim 1, wherein the cationic agent is chosen from cationic polymers and cationic surfactants.

33. The composition according to claim 32, wherein the cationic agent is a cationic polymer chosen from quaternary cellulose ether derivatives, cationic cyclopolymers, quaternary polymers of vinylpyrrolidone and of vinylimidazole, crosslinked homopolymers or copolymers of methacryloyloxy(C₁-C₄)alkyltri(C₁-C₄)alkylammonium salts, and mixtures thereof.

34. The composition according to claim 33, wherein the cationic cyclopolymers are chosen from dimethyldiallylammonium chloride homopolymers and copolymers.

35. The composition according to claim 1, wherein the cationic agent is present in a concentration ranging from 0.001% to 20% by weight relative to the total weight of the composition.

36. The composition according to claim 35, wherein the cationic agent is present in a concentration ranging from 0.05% to 10% by weight relative to the total weight of the composition.

37. The composition according to claim 36, wherein the cationic agent is present in a concentration ranging from 0.1% to 5% by weight relative to the total weight of the composition.

38. The composition according to claim 1, wherein the cationic agent is chosen from optionally polyoxyalkylenated primary, secondary and tertiary fatty amines, quaternary ammonium salts, and mixtures thereof.

39. The composition according to claim 38, wherein the cationic agent is a quaternary ammonium salt chosen from:
quaternary ammonium salt of formula (V): 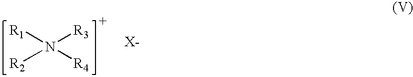
wherein R₁, R₂, R₃ and R₄, which may be identical or different, are chosen from linear and branched aliphatic radicals having from 1 to 30 carbon atoms, aromatic radicals; and
X⁻ is an anion chosen from halides, phosphates, acetates, lactates, (C₂-C₆) alkyl sulfates, and alkyl- and alkylaryl-sulfonates;
quaternary ammonium salts of imidazoline;
diquaternary ammonium salts of formula (VII): 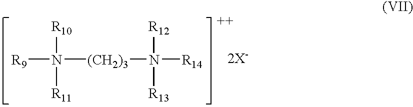
wherein R₉ is an aliphatic radical having from about 16 to 30 carbon atoms;
R₁₀, R₁₁, R₁₂, R₁₃ and R₁₄, which may be identical or different, are chosen from hydrogen and an alkyl radical having from 1 to 4 carbon atoms, and
X⁻ is an anion chosen from halides, phosphates, acetates, nitrates and methyl sulfates; and
quaternary ammonium salts comprising at least one ester function.

40. The composition according to claim 39, wherein the cationic agent is a quaternary ammonium salt of formula (V), where R₁, R₂, R₃ and R₄ are chosen from aryl and alkylaryl aromatic radicals.

41. The composition according to claim 38, wherein the cationic agent is a quaternary ammonium salt of imidazoline of formula (VI): 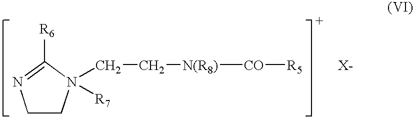
wherein R₅ is chosen from alkenyl and alkyl radicals having from 8 to 30 carbon atoms;
R₆ is chosen from a hydrogen atom, a C₁-C₄ alkyl radical, and alkenyl and alkyl radicals having from 8 to 30 carbon atoms;
R₇ is a C₁-C₄ alkyl radical;
R₈ is chosen from a hydrogen atom and a C₁-C₄ alkyl radical; and
X is an anion chosen from halides, phosphates, acetates, lactates, alkyl sulfates, alkyl sulfonates, and alkylaryl sulfonates.

42. The composition according to claim 41, wherein R₅ and R₆ are chosen from a mixture of alkenyl and alkyl radicals having from 12 to 21 carbon atoms.

43. The composition according to claim 42, wherein R₅ is a fatty acid derivative of tallow.

44. The composition according to claim 43, wherein R₆ is a fatty acid derivative of tallow.

45. The composition according to claim 41, wherein R₇ is methyl.

46. The composition according to claim 41, wherein R₈ is hydrogen.

47. The composition according to claim 1, wherein the cationic agent is chosen from behenyltrimethylammonium chloride, cetyltrimethylammonium chloride, quaternium-83, behenylamidopropyl 2,3-dihydroxypropyl dimethyl ammonium chloride, and palmitylamidopropyltrimethylammonium chloride.

48. The composition according to claim 1, wherein the cationic agent is present in a concentration ranging from 0.05% to 10% by weight relative to the total weight of the composition.

49. The composition according to claim 48, wherein the cationic agent is present in a concentration ranging from 0.1% to 8% by weight relative to the total weight of the composition.

50. The composition according to claim 1, further comprising at least one conditioning agent.

51. The composition according to claim 1, wherein the cosmetically acceptable aqueous medium comprises water or a mixture of water and of one cosmetically acceptable solvent.

52. The composition according to claim 1, wherein the cosmetically acceptable aqueous medium is chosen from C₁-C₄ lower alcohols, alkylene glycols, C₅-C₁₀ alkanes, and mixtures thereof.

53. The composition according to claim 52, wherein the cosmetically acceptable aqueous medium is chosen from ethanol, isopropanol, tert-butanol, n-butanol, propylene glycol, polyol ethers, and mixtures thereof.

54. The composition according to claim 1, further comprising at least one additive.

55. The composition according to claim 54, wherein the at least one additive is chosen from anionic, nonionic and amphoteric polymers; thickeners; opacifiers; nacreous agents; vitamins; provitamins; waxes; natural and synthetic ceramides; fragrances; dyes; organic and mineral particles; preserving agents; and pH stabilizers.

56. The composition according to claim 55, wherein the thickeners comprise acids and electrolytes, the provitamins comprise panthenol, and the waxes comprise plant waxes.

57. The composition according to claim 1, in a form chosen from a rinse-out or leave-in conditioner; a permanent-waving, relaxing, dyeing or bleaching composition; and an optionally rinse-out composition, to be applied before or after dyeing, bleaching, permanent-waving or relaxing hair or between the two steps of a permanent-waving or hair relaxing operation.

58. The composition according to claim 1, in the form of a rinse-out conditioner.

59. A cosmetic composition comprising, in a cosmetically acceptable aqueous medium:
at least one cationic agent chosen from cationic surfactants;
at least one oil; and
at least one polymer with a weight-average molecular mass of less than or equal to 1,000,000, comprising (a) a polymer skeleton comprising hydrocarbon-based repeating units comprising at least one heteroatom, and (b) at least one pendant or terminal fatty chain which is optionally functionalized, having from 6 to 120 carbon atoms and linked to the hydrocarbon-based units.

60. The composition according to claim 59, wherein the at least one oil is present in an amount ranging from 0.005% to 30% by weight relative to the total weight of the composition.

61. The composition according to claim 60, wherein the at least one oil is present in an amount ranging from 0.01% to 15% by weight relative to the total weight of the composition.

62. A process for treating a keratin material comprising:
applying to the keratin material, in an effective amount to treat the keratin material, a cosmetic composition comprising, in a cosmetically acceptable aqueous medium:
at least one cationic agent;
from 0.005% to 5% by weight of at least one oil;
at least one polymer with a weight-average molecular mass of less than or equal to 1,000,000, comprising (a) a polymer skeleton comprising hydrocarbon-based repeating units comprising at least one heteroatom, and (b) at least one pendant or terminal fatty chain which is optionally functionalized, having from 6 to 120 carbon atoms and linked to the hydrocarbon-based units; and
from 75% to 98% by weight of water relative to the total weight of the composition; and
optionally rinsing out the cosmetic composition.

63. The process of claim 62, wherein the keratin material is hair.
